(19) United States
(12) Patent Application Publication
(10) Pub. No.: US 2003/0225369 A1
(43) Pub. Date: Dec. 4, 2003

(54) Low profile transpyloric jejunostomy system

(75) Inventors: Donald J. McMichael, South Jordan, UT (US); Mark Elliott Foster, Sandy, UT (US); Dennis Cox, Sandy, UT (US); Kelly Jay Christian, Draper, UT (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/159,514

(22) Filed: May 31, 2002

(51) Int. Cl. A61M 29/00

(52) U.S. Cl. 604/104; 604/910

(57) ABSTRACT

The present invention relates to a low profile device having a head, a catheter segment, and a retention mechanism. The head of the catheter has at least two openings through which fluid may pass to or from a patient, the head having a low profile relative to a patient when the catheter is properly positioned in the patient. The catheter has at least a first and second lumen, each lumen being in communication with at least one of the openings of the head. The openings in the head of the catheter are designed to allow for the passage of fluid into the catheter and/or out of the patient. The low profile head may contribute to a reduction in the number of removals or displacements of as compared to traditional “on-low profile” apparatus in that the amount or length of the catheter or system which extends external the patients body is significantly less.

### BACKGROUND OF THE INVENTION

The present invention relates generally to enteral tubes, and more particularly to a low profile transpyloric jejunostomy balloon catheter having a low profile and methods to enable placement of the low profile devices.

There are numerous situations in which a body cavity must be catheterized to achieve a desired medical goal. One relatively common situation in which a body cavity is catheterized is to provide nutritional solutions or medicines directly into the stomach or intestines. A stoma is formed in the stomach or intestinal wall and a catheter is placed through the stoma. Feeding solutions can be injected through the catheter to provide nutrients directly to the stomach or intestines (known as enteral feeding). Additionally, with the use of enteral feeding catheters, for example, it is generally necessary to ensure that the catheter is not accidentally dislodged or removed from the stomach or intestines. This is true both during the actual administration or removal of fluids as well as the time periods in between feedings.

To ensure that the catheter is maintained in the proper position, it is common to use a balloon disposed along the catheter shaft. Inflating the balloon causes the balloon to contact the anatomical structure (i.e., a duct or stomach wall) and thereby prevent the catheter from moving out of the proper position. Depending on the type of catheter, the balloon may be positioned in a variety of locations along the catheter shaft. For example, with a G-tube the balloon will generally be at or near the distal end of the catheter, although the balloon or other retention mechanism may be slightly closer to the head of the catheter provided that the retention effect may still be achieved. Such balloon catheter devices may include a “low-profile” head at the proximal end of the catheter shaft. The head, which may help hold the balloon catheter in place, includes an opening for receiving the feeding solution and a one-way valve for preventing fluids from passing out of the patient via the catheter. U.S. Pat. Nos. 5,997,503 and 5,997,546, both owned by Applicants' Assignee and incorporated in their entirety by reference herein, disclose examples of low-profile balloon catheters suitable for enteral feeding.

As indicated above, there are a variety of instances in which it may be necessary to use a catheter, one of which is the not uncommon reaction following major surgery for one's stomach function to be impaired for a period of time. For instance, in addition to the need to supply or supplement the body with a certain level of nutrients and the like, following surgery, after an accident, as well as in other instances of impaired or limited gastric functionality, an unfed gut can become a source of bacteria that gets into the bloodstream. These types of problems may be resolved by the introduction of nutrients through a jejuneal tube properly inserted through the abdominal wall, gastric wall, pylorous, duodenum of a patient, into the jejunum beyond the Ligament of Treitz.

Transpyloric passage of a jejunal tube is technically difficult to achieve because of at least the following anatomic problems. First, the pylorus and duodenum are in the retro-peritoneum covered by small bowel mesentery and the right colon, making them poorly accessible to the surgeon's fingers. Secondly, the mucosal folds of the duodenum is redundant and creates ridges that prevent easy passage of a catheter.

While prior devices that are designed to span the pylorus are known to exist, each have certain shortcomings. For example, the Moss dual lumen gastrostomy tube, as described in Moss, U.S. Pat. Nos. 4,543,089 and 4,642,092, is intended for transpyloric feeding; however, the tube does not go beyond the duodenum. Since the tip of the Moss tube is situated just beyond the pylorus, nutrients from the tube tend to go back to the stomach rather than moving on to the jejunum. Accordingly, the Moss tube has proved of little benefit to critically ill patients who have had major surgical illness.

The Nyhus-Nelson system is a tube with two balloons that allow the surgeon to do a “push-me/pull-me” technique of passing a tube through the duodenum. This system has the disadvantage of being very time consuming for the surgeon and having a relatively high rate of failure.

Cook Incorporated markets a Carey-Alzate-Coons double lumen gastrojejunostomy set, as described in U.S. Pat. No. 4,581,025 and Re. 31,855, wherein the catheter is advanced over a wire guide after insertion of the wire guide through the pylorus and into the duodenum. The Cook device is designed for placement by a radiologist or gastroenterologist with access to an x-ray machine but is not suited for use at the operating table by a surgeon during major abdominal surgery.

Referring to the illustrative drawing of FIG. 1, there is shown a perspective view of an earlier enteral feeding device 20. The device 20 includes an elongated tubular member 51 formed from a stretchable elastomeric material such as silicone. FIG. 1A is an illustrative cross-sectional view of the tubular member 51 of the earlier device. The member 51 defines a jejunal tube 22, a gastronony tube 34 and a fluid line 46.

The jejunal feeding tube 22 includes an outlet end portion 24 which can extend through a patient's stomach into the jejunum. The jejunal tube outlet end portion includes perforations 26 which permit liquid food or medication to pass therethrough. The tube 22 is integrally connected to a jejunal tube inlet end portion 28 which defines a jejunal inlet port 30 having a removable plug cover 32.

The gastrostomy tube 34 is shorter than the jejunal tube 22 and includes a plurality of drainage inlets or food outlet ports such as inlet/outlet 36. A gastrostomy tube end portion 37 defines a gastrostomy inlet port 38 having a plug cover 40.

An inflatable balloon 42 is provided near the end of the gastrostomy tube 34 and is inflatable through a valve 44. The valve 44 is used to supply fluid to the balloon 42 through the fluid line 46.

Frictional contact between the elongated tubular member 51 and a locking/retention ring 56 is sufficiently great to prevent the member 51 from moving further into the stomach. The locking ring 56 remains in contact with a patient's abdominal wall during use. However, the frictional contact also is sufficiently low to permit adjustment or placement of the member 51 relative to a patients abdomen.

Referring to the illustrative drawings of FIG. 2, there is shown a perspective view of an earlier device 20 in use. The inflated balloon 42 forms a gasket that seals the entrance to the stomach, and together with the locking ring 56 secures the device 20 in place.

While prior feeding tubes generally have been able to accomplish the desired task of providing nutrition to a patient, there have been shortcomings with their use. In particular, transpyloric jejunal (also referred to as gastrojejunal) tubes were originally designed for use with non-ambulatory patients. As such, it was not previously important how long or far the tube or lumens extended from or out from the patient. Previously, it was not uncommon for large external quantities of tubing to be present. However, as experienced with a number of different patients, for example, combative or ambulatory patients, the length of tubing which extends from the patient was deemed not only unnecessary, but detrimental as it has a tendency to get tangled or caught on something. In such instances the chance of dislodging the tube or rendering the tube ineffective was high, and thereby significantly increased the chance that the tube insertion procedure would need to be repeated as well as the possibility that the tube could cause damage when the dislodged. Additionally in ambulatory patients, there is the potential for cosmetic embarrassment, in that it was previously difficult to hide the presence of the tubing from others when access to the tubing was not necessary or required. Even when the patient is non-ambulatory, in some situations (e.g. children and/or patients prone to seizure), it would be desirable to have the option of a low profile tube.

Attempts have been made to provide a low-profile feeding tube, such as U.S. Pat. No. 5,073,166 to Parks et al. and U.S. Pat. No. 6,264,631 to Willis et al., both of which are commonly owned by the Assignee of the present invention, and both of which are incorporated by reference in their entirety. However, these devices are not intended for transpyloric use. WO 01/60313, assigned to Sherwood Services, also makes an attempt to address some of the concerns with non-low-profile feeding devices, via the disclosure of an adapter and a system which uses the adapter, however, devices disclosed therein require the person inserting the adapter to feed a feeding tube through the adapter before the adapter can be used and to undertake several other preparation steps (some post insertion) before the adapter or the system could be considered low profile.

Thus, while a number of improvements have been made to conventional enteral tubes, there exists the need and the desire to produce a low-profile TJ tube or system which is complete and/or does not require on-site assembly or point of use construction. Generally, the users of TJ tubes would also benefit (e.g. reduced dislodgements and/or improvement in the ability to reduce cosmetic embarrassment and the like) from a low-profile article.

### SUMMARY OF THE INVENTION

In response to the difficulties and problems discussed above, a new low profile catheter has been developed. The present invention relates to a catheter or system for providing nutrition and/or medication to a patient whose stomach function is inhibited or nonfunctional. More particularly, the present invention relates to feeding tubes or feeding devices, such as transpyloric feeding tubes and the like, which are adapted to enable the provision of nutrition and/or medicine directly into the patient's jejunum where the patient is experiencing a nonfunctioning or impaired stomach and/or difficulties swallowing, chewing or the like. The devices contemplated by the present invention may also permit nutrients to be placed directly into a patient's stomach and/or into the patient's jejunum. As noted above, this may be necessary when a patient has a disorder of the gastrointestinal tract, malabsorption (impaired absorption of nutrients, vitamins or minerals from the diet by the lining of the small intestine), or neurological or renal disorders.

More specifically one aspect of the present invention relates to a catheter having a head, a catheter segment, and a retention mechanism. The head of the catheter has at least two openings through which fluid (including gases) may pass to or from a patient, the head having a low profile relative to a patient when the catheter is properly positioned in the patient. The catheter segment extends from the head of the catheter and has a proximal end and a distal end. The catheter has at least a first and second lumen, each lumen being in communication with at least one of the openings of the head. The openings in the head of the catheter are designed to allow for the passage of fluid, including fluids with solids, into the catheter and/or out of the patient. The low profile head may contribute to a reduction in the number of accidental removals or displacements of the feeding tubes or systems as compared to traditional “non-low profile” apparatus in that the amount or length of the catheter or system which extends external the patient's body is significantly less. Additionally, the improved cosmetic appearance of a low profile system or device is desirable for many patients, especially those who are ambulatory and/or those individuals who would like to be able to conceal the fact that they have an enteral feeding tube.

In one of the embodiments of the present invention, the retention mechanism may be a balloon. Specifically, the balloon may be formed by a sleeve with a first end attached to the catheter segment so as to form a first cuff and a second end attached to the catheter segment so as to form a second cuff, the balloon collapsing on the catheter segment when not inflated.

For purposes of this application, unless the context demands a different meaning, or a different meaning is expressed, the term “stoma” is intended to include fully formed stomas (as will be appreciated by those in the art) as well as any other opening or aperture or the like (including, for example, punctures, holes, passages or the like through, for example, the abdominal wall or gastric wall of a patient), even if newly created and/or not fully formed or healed (as with conventional stomas), through which the device of the present invention may be passed in accordance with the description herein so as to enable the uses contemplated by the present invention. References to the passage of an item through the abdominal or gastric wall of a patient is intended to include not only initial insertions or the creation of an opening, puncture, or the like so as to enable the passage of the item therethrough, but also passage of the item or items through a existing opening (e.g. an existing puncture or an existing stoma). As used herein, unless the context demands a different meaning, or a different meaning is expressed, the term “patient” is intended to include any and all such patients and is not limited to humans, but may also include, for example, other creatures such as animals, mammals and the like. As used herein, unless the context demands a different meaning, or a different meaning is expressed, the terms “puncture” or “puncturing” when used as a verb is intended to include all manners of creating an opening, hole, aperture or the like, including, but not limited to puncturing, piercing, cutting, slitting and the like. As used herein, unless the context demands a different meaning, or a different meaning is expressed, the term “feed tube” means any and all suitable devices which may be used in accordance with the present invention to provide food, nutrition, medicine, fluids and/or the like therethrough. An example of a suitable “feed tube” is a catheter, and more specifically a transjejunal catheter.

Another embodiment of the present invention relates to a balloon catheter adapted for placement through a stoma into a body cavity so that the balloon catheter is maintained in the stoma. The balloon catheter includes a head having at least two openings through which a fluid may be introduced or passed, the head being low profile relative to a patient when the catheter is correctly positioned within the patient; a catheter segment extending from the head to a distal tip of the catheter, the catheter segment having an exterior and a wall defining at least one passageway through an interior; and an elongate sleeve attached to the exterior of the catheter segment about the passageway so as to form an inflatable balloon which covers a portion of the catheter segment. The sleeve may preferably be formed such that in its collapsed state (i.e. when not inflated) the sleeve closely surrounds the catheter segment, and when inflated the sleeve extends radially outwardly from the catheter segment. In this embodiment, the inflation of the sleeve may be controlled through one of the openings in the head of catheter as the sleeve is in communication with one of the openings in the head of the balloon catheter.

Yet another embodiment of the present invention defines a balloon catheter adapted for placement through a stoma into a body cavity so that the balloon catheter is maintained in the stoma. The balloon catheter includes a low profile head having at least three openings through which a fluid may be injected, the head having a low profile relative to a patients body when the catheter is correctly positioned; a catheter segment extending from the head to a distal tip, the catheter segment having an exterior and a wall defining a passageway through the interior, the passageway defining at least three lumens, each lumen having a proximal end and a distal end; and an elongate sleeve attached to the catheter segment about the passageway so as to form an inflatable balloon which covers a portion of the exterior of the catheter segment, the elongate sleeve being in communication with one of the at least three lumens, the sleeve being in collapsed state when not inflated wherein the sleeve closely surrounds the catheter segment, and wherein the sleeve extends radially outwardly from the catheter segment when inflated.

One embodiment of the present invention is also directed to a low profile transjejunal feeding system. The feeding system includes a balloon catheter having multiple lumens, a low profile head having at least two ports therein, and a balloon retention mechanism; and a first adapter capable of attachment to one of the ports of the balloon catheter; wherein each of the lumens is in communication with at least one of the ports.

The invention will be more fully understood and further advantages will become apparent when reference is made to the following detailed description of the invention and the accompanying drawings.

### BRIEF DESCRIPTION OF THE DRAWINGS

FIG. 1 is a perspective view of an earlier feeding tube.

FIG. 1A is a cross-sectional view along line 1A-1A of FIG. 1 illustrating the disposition of the jejunal and gastrostomy tubes and the connecting line.

FIG. 2 is a perspective partially cutaway view of an earlier feeding tube installed in a patient.

FIG. 3 is a perspective view of one embodiment of a catheter of the present invention.

FIG. 3A is an enlarged perspective view of an alternate embodiment of the distal portion of the catheter shown in FIG. 3.

FIG. 3B is a perspective view of alternate embodiment of the catheter shown in FIG. 3, wherein the catheter in FIG. 3B does not show a retention mechanism and does not have the optional distal tip.

FIG. 4 is an overhead view of the head of the catheter shown in FIG. 3.

FIG. 4A is a cross-sectional view of the catheter of FIG. 4, taken along line A-A.

FIG. 4B is a cross-sectional view of the catheter of FIG. 4, taken along line A-A, illustrating the presence of an optional guide wire.

FIG. 4C is a enlarged cross-sectional view along line 4B-4B of FIG. 3 illustrating the orientation of the lumens of the catheter in one embodiment.

FIG. 5 is a perspective view of an alternate embodiment of the head of a device of the present invention.

FIG. 5A is a perspective view of another embodiment of the head of a device of the present invention.

FIGS. 6 and 6A are perspectives view of the device of FIG. 3 showing the retention mechanism in an inflated or expanded position.

FIG. 7 is a cross-sectional view of one embodiment of an adapter suitable for use with the present invention.

FIG. 7A is a cross-sectional view of one embodiment of another adapter suitable for use with the present invention.

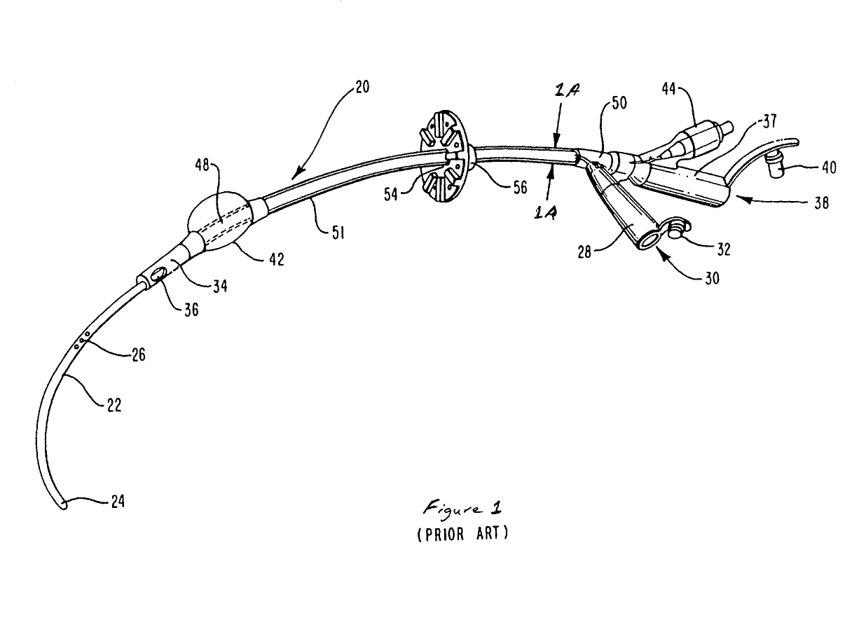

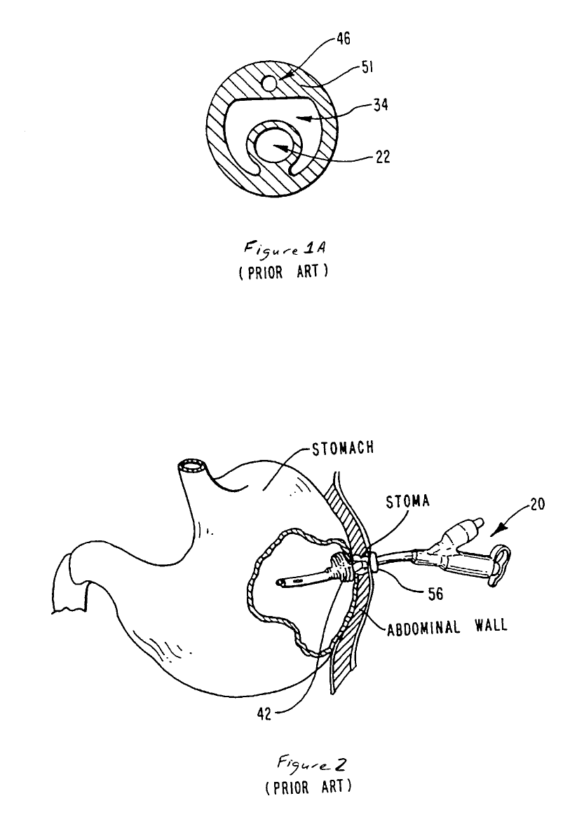

### DETAILED DESCRIPTION OF THE INVENTION

The following detailed description will be made in the context of a low profile catheter or transjejunal tube. It is readily apparent, however, that the article of the present invention would also be suitable for use as other types of catheter products and devices and the like. In addition, the invention will be described in the context of its various configurations. It should be appreciated that alternative arrangements of the invention can comprise any combination of such configurations. As such, the use of a preferred embodiment, a balloon catheter, for ease in understanding and describing the invention shall not, in any manner, limit the scope of the invention.

It is of note that the term “transjejunal” is frequently interchanged with the terms “transgastro-jejunal” and/or “gastrojejunal”. While the term “transjejunal” is used throughout this disclosure, the term is intended to include the terms “transgastro-jejunal” and “gastro-jejunal” and/or any other term known to those having skill in the art which has a similar or an equivalent meaning.

The articles of present invention advantageously exhibit a low profile relative to the patient, and more preferably relative to the abdomen of the patient, when the article is properly positioned within a patient. The low profile attributes results in a reduction in the amount of the device which is positioned external to the patient as compared with a traditional or non-low profile device. The reduction in tubing and/or other portions generally found external to the patient in low profile devices as compared with non-low profile devices should provide for a reduction in the number of accidental displacements or removals of the articles. This will be true because the reduction in exposed tubing and/or size of the head attached thereto reduces the likelihood that a patient, especially a patient who is ambulatory or one who is active or prone to seizures, may somehow catch, snag, or entangle the protruding portion of the device in clothing, furniture, or the like thereby resulting in the unintentional or undesired dislodgement or removal of the device. Additionally, the low profile characteristics of the device may also provide improved or more preferable aesthetic and cosmetic characteristics for the device in that many patients, especially those who are ambulatory and who do not wish others to know about their condition or the presence of an enteral feeding device, will appreciate that the low profile head allows for a patient to more readily hide the protruding portion of the device below clothing.

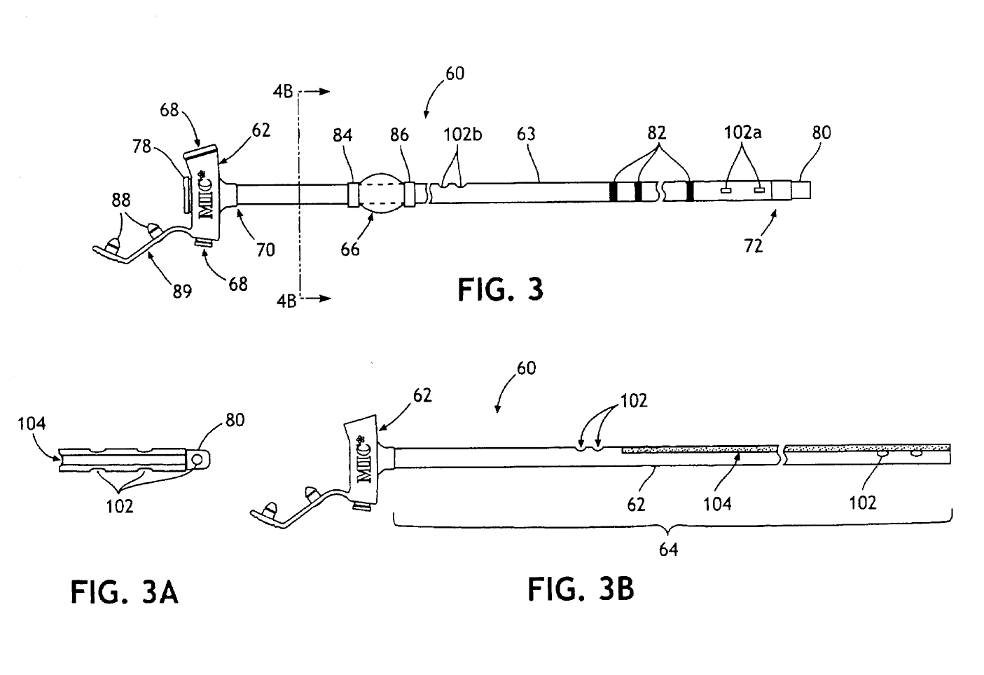

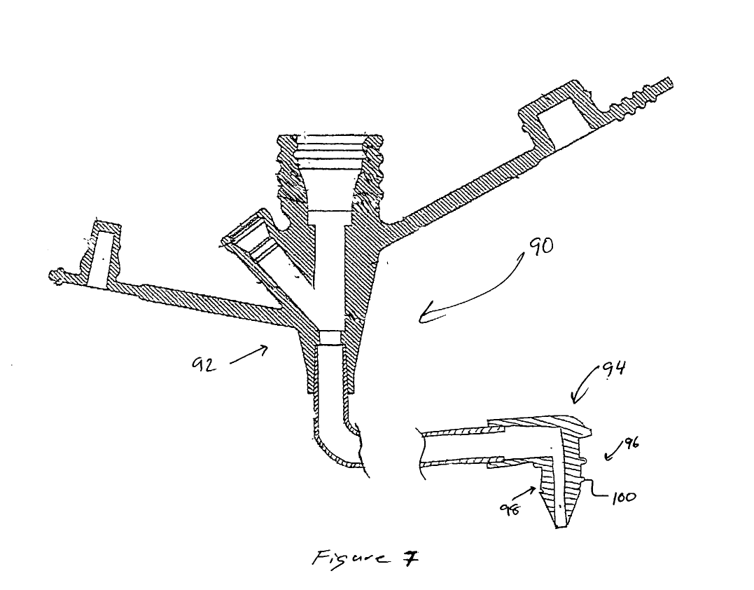

Referring now to the FIGS. 3-6A, the present invention provides a catheter 60 having a head 62, a catheter segment 64, and a retention mechanism 66. The head of the catheter has at least two openings 68a and 68b through which fluid may pass to or from a patient, the head 62 having a low profile relative to a patient when the catheter 60 is properly positioned in the patient. As noted above the catheter of the present invention is preferably situated such that the head is positioned adjacent the abdomen of the patient, however, it may be preferable in some circumstances for the head 62 of the catheter to be positioned elsewhere on the patient. The catheter segment 64 extends from the head 62 of the catheter and has a proximal end 70 and a distal end 72. The catheter 60 also has at least a first lumen 86a and second lumen 86b, each of the lumens having a proximal end 74a, 74b and a distal end (not shown), and each lumen being in communication with at least one of the openings 68 of the head 62. The openings 68 in the head of the catheter are designed to allow for the passage of fluid into the catheter 60 and/or out of the patient. Depending on the embodiment of the invention, one or more of the openings 68 may also preferably include a valve 78 configured for selectively controlling fluid flow through one or more of the lumens of the catheter. Suitable valves 78 include, for example, one-way, two-way, and duckbill valves. The valves 78 can assist with the regulation of fluids to and/or from the patient. For instance, it may be preferable for one or more of the openings 68 in communication with the lumen or lumens which are used to provide nutrients, medicine and/or other fluids to the patient (as discussed below) to contain a one-way valve, such that the fluid or fluids intended to pass into the patient cannot or do not flow back out through the head of the catheter or so that fluids from within the patient are not unintentionally allowed to flow out of the catheter or at least certain lumens of the catheter. Alternately, an opening in the head 62 of the catheter 60 which is in communication with the stomach or gastric cavity of a patient may have a valve which permits fluid to flow through the head 62 and into the patient as well as to permit flow of a fluid from the patient into the catheter 60 and out an opening 68 (e.g. venting), as discussed in more detail below. Further, where the retention mechanism is a balloon or elastomeric sleeve, as discussed below, it may be preferable for the opening 68 to include a one way check valve which can assist with inflation and/or deflation of the balloon as well as maintaining the preferred level of inflation.

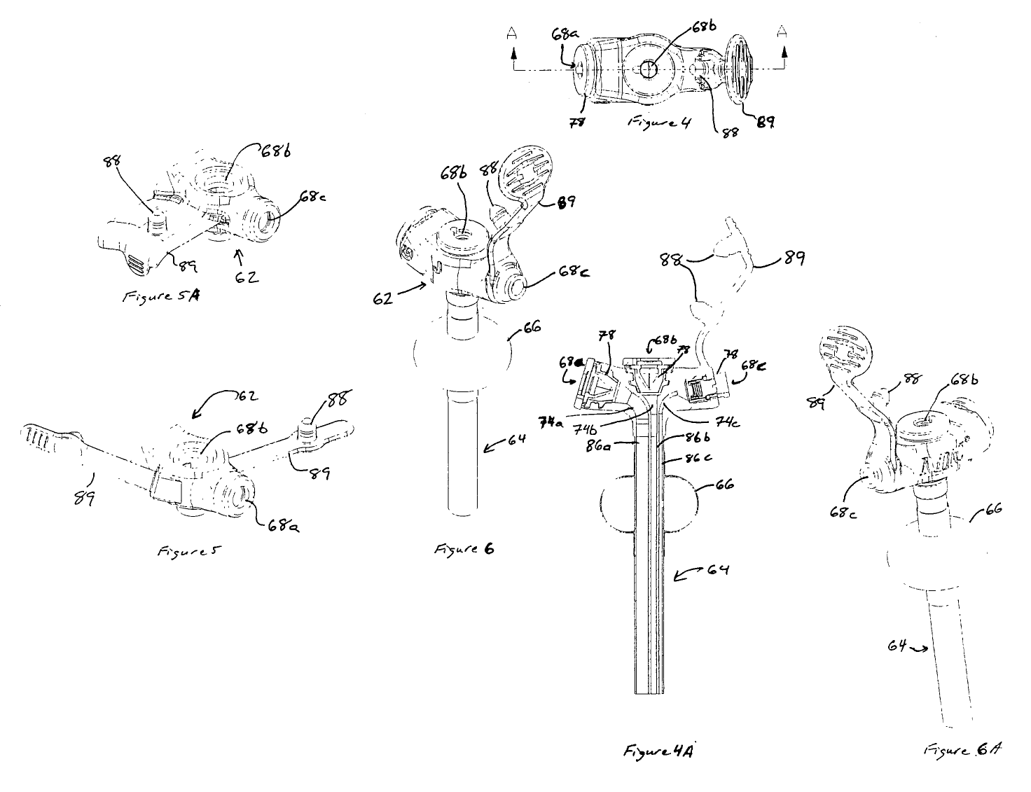

Each of the lumens of the catheter 60 includes an entry port (not shown) in the proximal portion of the lumen and an exit port (not shown) in the distal portion of the lumen. More specifically depending on the formation of the head and lumens of the catheter, the entry port of the lumen may be one of the openings 68 in the head 62 of the catheter. Whether the lumens are integrally formed with the head of the catheter (as illustrated in FIG. 4A) or require a connection thereto, each lumen 86 in the catheter should be in communication with at least one opening 68 of the head 62.

Preferably, the distal end 72 of the catheter segment 64 may be adapted to terminate within the jejunum of a patient; however, the ultimate location or positioning of the catheter, including its distal end 72, will vary from patient to patient and will depend to some degree on the method of insertion or placement as well as the person who places the catheter in the patient. As such, in use the distal end 72 of the catheter segment 64 need not be placed, or it may be undesirable to place, the distal end such that it terminates within the jejunum of the patient, but the devices of the present invention have that capability. That is in use, although not the preferred use, the distal end of the devices of the present invention may not be placed or positioned so as to terminate in the jejunum, but rather may terminate in the small intestine of the patient. It is appreciated that people are of various sizes and weights and as such the location of certain body parts will not be the same from person to person. Accordingly, the present invention may be manufactured in a variety of sizes and lengths, such that patients of all sizes can be accommodated. For example, low volume balloons may be used on pediatric or pediatric sized catheters. All dimensions of the devices are envisioned to be variable and/or to have components that are scalable collectively or individually.

To assist with the placement of the devices of the present invention, the catheter segment 64 preferably includes a catheter shaft 63 having a distal end 72 and a stiff tip 80 attached to the distal end of the catheter shaft. In addition to providing a stiff tip 80 which can assist or enable penetration of certain portions of the patient's anatomy such as the Ligament of Treitz and the duodenum, the stiff tip 80 may also provide rigidity which can assist in the placement of the distal end 72. Furthermore, the stiff tip 80 can act to resist collapse or crushing of the end of the catheter, thereby preventing or reducing the likelihood of blockage or clogging of the catheter. The tip 80 is preferably rounded and has a soft tip to prevent or reduce abrasion. The catheter 60 may also include markers or markings 82 along the catheter segment 64, which may assist the person placing the device. The markings 82 may be visible without the assistance of other devices, (e.g. printed on the exterior of the catheter segment) or may require other means to view (e.g. radiopaque markers visible in x-rays) as discussed below.

In the present invention, a retention mechanism 66 is used to assist with the positioning of the catheter 60. Once activated or triggered, the retention mechanism 66 serves to keep the catheter in place such that it is not readily removed or dislodged from the patient. The retention mechanism 66 generally extends or expands in such a way that the catheter 60 cannot be readily withdrawn until the mechanism is withdrawn or deflated. When the mechanism is in its withdrawn or deflated state, as shown in FIG. 3, the catheter is able to be readily moved in and out of the stoma through which it is inserted. The retention mechanism 66 is located along the catheter segment 64 at a position between the proximal end 70 and the distal end 72. Any location along the catheter segment 64 is suitable for the retention mechanism 66 provided it allows the desired retention to occur. Preferably, the retention mechanism 66 is positioned along the catheter segment 64 such that when engaged, triggered, inflated, or the like, the retention mechanism will be positioned adjacent to the abdominal wall of the patient. Alternatively, depending on the type of mechanism being used, it may be preferable for the retention mechanism to be positioned along the catheter segment such that when activated, the mechanism comes in contact with a duct within the patient in such a manner so as to create the desired retention function. Furthermore, when used within a duct within a patient, the retention mechanism 66, may also act to seal off the duct to prevent or reduce backflow through the duct. As one skilled in the art, any suitable retention mechanism may be used with the present invention, however, depending on the portion of the patient which the mechanism may contact, certain types of mechanisms may be more suitable than others. Several types of retention mechanisms are known to those skilled in the art. Suitable examples include, but are not limited to, those disclosed in U.S. Pat. Nos. 5,073,166 to Parks et al. and 6,264,631 to Willis et al.

While any suitable retention mechanism will work, it may be preferable to use a balloon or an elastomeric sleeve 66 as the retention mechanism. Preferably, such a balloon 66 may be formed by a sleeve with a first end attached to the exterior of the catheter segment 64 so as to form a first cuff 84 and a second end attached to the exterior of the catheter segment so as to form a second cuff 86, the balloon 66 collapsing on the catheter segment 64 when not inflated to thereby facilitate insertion or removal through a stoma. Although, it may be preferable for the sleeve or balloon 66 to be attached to the exterior of the catheter segment 64, it is contemplated that in one or more embodiments one or both ends of the sleeve or balloon may be secured to the interior of the catheter segment 64. Regardless of the type of retention mechanism used or the placement of the retention mechanism within the patient (e.g. against the stomach wall or in a duct), it is preferred that the retention mechanism hold the catheter in the place such that the low profile head generally rests against the body of the patient, and more preferably in a close or snug fit with or adjacent to the abdomen of the patient, but preferably not so close or snug so as to cause discomfort to the patient as a result of squeezing or pinching.

Another preferred characteristic of the catheter 60 of the present invention is that the low profile head 62 may have three openings 68a, 68b, and 68c (as shown in FIG. 4A) and the catheter segment 64 may have three lumens 86a, 86b, and 86c (as shown in FIG. 4A). Referring now to the catheter 60 shown in FIG. 4A, the low profile head 62 includes a first opening 68a for communication with a lumen 86a intended for fluid communication between the low profile head 62 and the jejunum of the patient; an opening 68b for communication with a lumen 86b intended for fluid communication between the low profile head 62 and a visceral organ of the patient; and an opening 68c for communication with a lumen 86c intended for communication between the low profile head 62 and the retention mechanism. While the catheter of FIG. 4A illustrates a low profile head 62 with three openings and a portion of catheter segment 64 having three lumens 86a, 86b, and 86c, the present invention contemplates and one skilled in the art will appreciate that additional openings and/or lumens may be included, limited only by the size, shape and intended use of the catheter.

In another embodiment, the low profile head 62 of the catheter 60 has a plurality of external surfaces and the openings 68 of the low profile head 62 may be on one or more surfaces of the low profile head. That is, depending on the shape and size of the head of the catheter, the openings in the head of the catheter may all be on one surface of head or the openings may be on multiple surfaces of the head. One skilled in the art will appreciate that the size and shape of the low profile head may vary, and that additional components (e.g. additional openings, etc.) may be included in those devices having larger heads. All variations in size and shape of the head of the catheter as well as the size, spacing and orientation of the openings around the head are contemplated by the present invention and are intended to be covered by and included in the claims of the present invention.

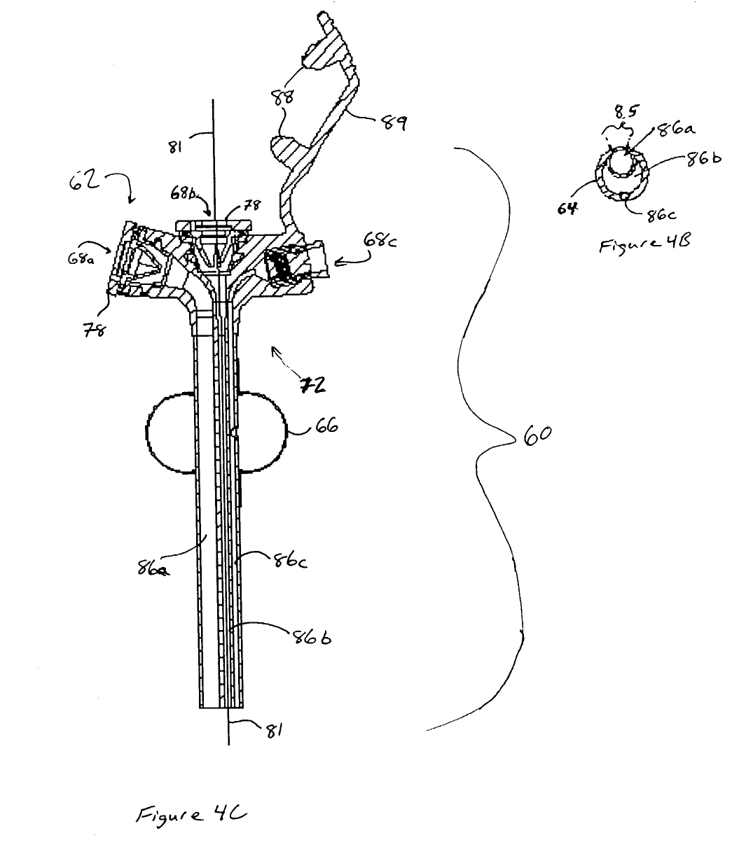

The present invention may further include a protective covering 88 (as shown in FIGS. 3, 3B, 4, 4A and 5-6A) for at least one of the openings 68. Any suitable protective covering 88 is acceptable. Examples of suitable protective coverings 88 include, but are not limited to, plugs, caps, inserts and the like. Some embodiments may include a protective covering which is attached to or formed as a part of a strap 89. Such a strap 89 may have one or more protective coverings 88 attached thereto or formed thereon depending on the design of the catheter head and the orientation of the openings in the head (see, for example, FIGS. 3 and 5A). Other embodiments may have multiple straps (see, for example, FIG. 5) or may not have any straps or protective coverings at all (not shown). Where multiple protective coverings 88 are included on one strap 89, the coverings will preferably be designed such that they may fit any of the openings 68 they may be inserted into. That is, it is preferable for the openings and/or the protective coverings to be universal in size and shape such that the coverings are interchangeable among the openings, limited only by the ability of a covering 88 to reach an opening 68 because of the length of the strap, cord or the like 89 to which the covering 88 is attached. It will be appreciated that the strap, cord or any other suitable attachment mechanism 89 may be formed integrally with a portion of the device of the present invention as shown, for example, in FIGS. 3 and 5, or the suitable attachment mechanism 89 may be separately secured or attached to the device of the present invention. That is, in one embodiment the proximal end of the catheter segment may include at least one external retention member or attachment mechanism 89 and a protective covering 88 formed with the external retention member 89. Further where the attachment mechanism or external retention member 89 is not formed integrally with the present invention, the external retention member may include a means for securing (not shown) the protective covering to the catheter. It is appreciated that the means for securing the protective covering may 88 or may not be integrally formed with mechanism or member 89.

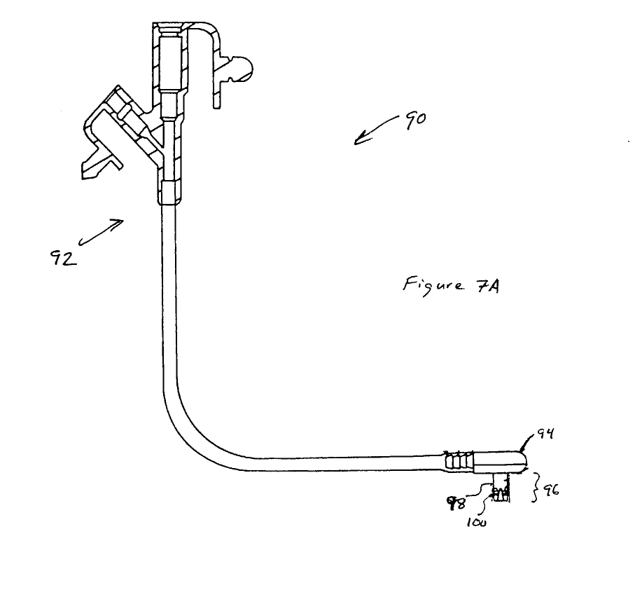

As noted above, the head 62 may be of different sizes and shapes. Additionally, depending on the size of the catheter 60 being used, one skilled in the art will recognize that the head 62 of the catheter 60 may have a larger cross-sectional diameter than the catheter segment 64. Further still, the head of the catheter of the present invention may be capable of receiving an adapter 90 (as shown in FIGS. 7 and 7A). That is, in at least one embodiment of the present invention, it may be preferable for at least one of the openings 68 of the low profile head 62 to be capable of receiving an adapter 90. The present invention may further include an adapter for communication with one or more of the lumens of the catheter with the adapter having a proximal end 92 and a distal end 94 and one or more lumens which allow fluid communication between the proximal end 92 and the distal end 94 of the adapter 90. Suitable exemplary adapters include, but are not limited to, those described and disclosed in U.S. Pat. Nos. 5,073,166 and 6,264,631 and U.S. patent application Ser. No. 09/660665, each of which is incorporated by reference in their entirety, as well as those known to those skilled in the art, such as feeding tubes, infusions sets, and the like. Those embodiments capable of receiving an adapter preferably have a positioning mechanism (not shown) capable of enabling proper alignment of the lumens in the catheter with the one or more lumens in the adapter. That is in those embodiments where the one or more openings in the head of the catheter is in communication with more than one lumen, the catheter may contain a means for enabling or ensuring that the lumens in the adapter are properly aligned with the lumens in communication with the opening to which the adapter is connected such that fluids intended to pass through one lumen of the catheter do so and are not inadvertently or unintentionally allowed to pass through one or more of the other lumens of the catheter.

Some of the advantages of the low profile aspects of the present invention are discussed above, however, the use of an adapter, extension sets or the like 90 may provide additional advantages. For example, in non-low profile devices, it is common for the portion of the device or apparatus which extends from the patient (e.g. that portion of the tube or catheter external of the patient's abdomen) to become clogged. While the reasoning is not known with specificity, it is has been observed that the portion of a feeding tube or catheter which extends beyond the patient has a greater incidence of clogging than the portion of the tube or catheter which is inside the patient. As such the need to replace the device comes about more frequently because of clogging external to the body than would be necessary because of clogging experienced in the portion of the tube or catheter inside the patient. As such the present invention will reduce the need for or extend the time between the replacement of the tube or catheter as the adapter(s), extension sets, and the like 90 which connect thereto may be readily removed, cleaned and replaced or replaced all together with a different adapter, extension set or the like may be removed and cleaned and then replaced. The reduction in the number of times the catheter must be replaced is highly preferred not only because of the reduction in the procedures, but also, for example, because of the reduction in exposure to trauma which the patient may experience because of the increased number of procedures.

Preferably the catheter 60 may also include a means for releasably latching the adapter 90 to the low profile head 62 of the catheter. Any suitable means for releasably latching the adapter will work and will be appreciated by those skilled in the art. As shown in FIG. 7A, an exemplary latching means 96 may include a leg 98 extending from the distal end 94 of the adapter 90 and a finger 100 formed at a free end of the leg 98 and extending at an angle generally perpendicular relative to the leg 98. The leg 98 and finger 100 serve to guide the adapter 90 into the latching means 96. Once inserted into the head of the catheter, the distal end 94 of the adapter 90 may, for example, be rotated such that the adapter 90 is retained in place until rotated back to the insertion position. In embodiments of the present invention where the adapter 90 contains multiple lumens the latching mechanism or means 96 may also serve or function to ensure proper alignment of the lumens of the adapter with the appropriate lumen or lumens of the catheter.

One skilled in the art will appreciate that each of the lumens 86a, 86b, 86c of the catheter 60 preferably has at least one opening or aperture (not shown) at or near the distal end of the lumen. It will be appreciated by those skilled in the art that where a lumen extends beyond the opening or aperture at or proximate the distal end of the lumen, but where the lumen is plugged, filled or blocked (e.g. where a radiopaque material fills the distal end of the lumen, as discussed below), the point at which the lumen is plugged, filled or blocked shall be considered the distal end of the lumen. (This is best illustrated in FIG. 3B, where apertures 102b are near the end of the portion of the gastric or venting lumen (not shown) through which fluid may pass as, in that embodiment, the radiopaque material 104 fills the portion of that lumen in the distal portion of the catheter so as to create a plug which blocks the flow of fluid through that lumen beyond the point at which the radiopaque material 104 is positioned). The apertures 102 at or near the distal end (not shown) of the lumens provide for the flow of fluids therethrough to or from the exterior of the catheter. It will also be appreciated that preferred catheter segments 64 will have at least one opening 102 (as shown in FIG. 3B) therein corresponding to the one or more of the openings or apertures at or near the distal end of the lumens. The opening or aperture 102 in the catheter segment 64 which corresponds with openings in the distal end (not shown) of the lumens 86 allows fluid to pass therethrough so as to complete the communication between the head 62 of the catheter 60 and the exterior of the catheter segment 64. For example, nutritional fluids may preferably pass through the head of the catheter, through one of the lumens and out of the distal end of the lumen into the desired location in the patient; or gas may, for example, be vented by passing from the patient into one of the lumens and out through the head of the catheter. For purposes of this disclosure, fluids such as saline, which are provided to trigger the retention mechanism 66 (e.g. inflate or expand the balloon or sleeve) shall also be considered to pass to the exterior of the catheter segment 64 as those fluids pass through the head 62 of the catheter 60 into the lumen and outside the catheter segment 64 as the mechanism 66 expands even though the fluid may never pass to or from the patient through the lumen in communication with the retention mechanism 66.

It will also be appreciated that multiple apertures or openings may be present in catheter segment 64 and/or the distal end of the lumens 86 such that fluid flow is not inhibited should one or more of the other apertures 102 in a lumen or the catheter segment (for example, as shown in region 85 of FIG. 4B, depending on the formation of the catheter, the wall of the catheter segment 64 may be both a wall of a lumen and a wall of the catheter segment such that a opening created in one is an opening created in the other) become clogged or blocked. Further still, it will be appreciated by those skilled in the art that the size, shape and spacing of the apertures in the lumens or catheter segment may vary and will depend in part on the desired application. All such variations are contemplated and intended to be included and covered by the present invention. For example, the number and/or size and/or location of apertures along a lumen or catheter segment may vary depending on the size (both diameter and length) of the catheter segment and lumens being used. That is, for example, the number and size of the apertures in the catheter segment in an adult catheter may not be the same as a pediatric catheter or even an adult catheter having different length catheter segments.

Depending on whether the catheter is to be placed during a surgery or a less traumatic fashion or to be used as a replacement for a pre-existing device, it may be preferable for the catheter to also include or be used in connection with a guide wire 81. During use or placement the wire may extend through the catheter, preferably through the head and a opening at the distal end 72 of the catheter (as shown in FIG. 4C). The catheter may be slid over the guide wire 81 to assist with the placement of the catheter, or in other embodiments the wire may be used to control or steer the distal end or distal tip (not shown in FIG. 4C) of the catheter 60 to better enable preferred placement of the device.

Yet other embodiments may comprise additional features or components, such as a radiopaque material or marker 104. The radiopaque material 104 may be positioned at one or more points along the catheter 60, but is preferably positioned at or near the distal portion of the catheter segment 64, such that the location of the distal end 72 of the catheter, and more particularly the distal tip 80, if present, may be verified by x-ray or other suitable means. The radiopaque material 104 may be used in a variety of forms, such as tubes, plugs, stripes, markers, tips, and the like with the preferred form depending on the application and the person placing the catheter. In some embodiments, it may be preferable to fill or substantially fill a portion of a lumen with a radiopaque material. Such an embodiment is shown in FIGS. 3A and 3B where the distal portion of the lumen having apertures is filled with tungsten (or a tungsten containing material or powder) 104, such that the tungsten provides the ability to determine the position of the portion of the catheter in which the tungsten is present without inhibiting or hindering the operational ability of the remainder of the catheter and more specifically without interfering with the apertures 102 along the catheter segment. One skilled in the art will recognize that there are a number of suitable radiopaque materials and that any suitable material may be used in the present invention. As noted above, an example of such a suitable material is tungsten or a tungsten containing powder or material.

The present invention is also directed to a balloon catheter adapted for placement through a stoma into a body cavity so that the balloon catheter is maintained in the stoma. The balloon catheter includes a head having at least two openings through which a fluid may be passed, the head being low profile relative to a patient, and preferably the patient's abdomen, when the catheter is correctly positioned within the patient; a catheter segment extending from the head to a distal tip, the catheter segment having an exterior and a wall defining at least two passageways through the interior; and an elongate sleeve attached to the exterior of the catheter segment about the passageway so as to form an inflatable balloon which covers a portion of the catheter segment. The sleeve is preferably formed such that in its collapsed state (i.e. when not inflated) the sleeve closely surrounds the catheter segment, and when inflated the sleeve extends radially outwardly from the catheter segment. In this embodiment, the inflation of the sleeve may be controlled through one of the openings in the head of catheter as the sleeve is in communication with one of the openings in the head of the balloon catheter. Preferably the passageway through the interior of the catheter segment has at least a plurality of lumens, at least one of which extends from the head of the catheter to a point at or near the distal end of the segment, and at least one of which extends from the head of the catheter to the elongate sleeve.

A preferred embodiment of the balloon catheter may also have one or more of the following attributes: the attachment of the elongate sleeve to the exterior of the catheter segment forms a proximal cuff and a distal cuff of the balloon; the catheter segment includes a catheter shaft and a stiff tip attached to a distal end of the catheter shaft, opposite the head; one of the lumens of the catheter is a feeding tube, the distal end thereof which is preferably adapted to terminate within a jejunum of a patient; one of the lumens may be a venting lumen, wherein fluid contained within a visceral organ of a patient may be evacuated through the venting lumen; and one or more of the openings of the head of the catheter may be capable of receiving an adapter. The ability to receive an adapter provides the patient the ability to temporarily attach or to allow connection to a feeding tube, infusion set or the like at desired times, while providing a more compact apparatus when not attached thereto, when a larger more conspicuous apparatus may be undesirable. For example, a larger more conspicuous apparatus or device may be undesirable when in public or if a patient is ambulatory or affected by seizures such that a longer extension is more likely to get caught on or become entangled in something, possibly resulting in the unintentional removal or partial removal of the device. As discussed in more detail below, the ability to use an adapter or the like also reduces the frequency at which the catheter needs to be replaced, as the adapters or other connections may be removed and cleaned or discarded and replaced.

Yet another embodiment of the present invention defines a balloon catheter adapted for placement through a stoma into a body cavity so that the balloon catheter is maintained in the stoma. The balloon catheter includes a low profile head having at least three openings through which a fluid may be injected, the head being low profile relative to a patient's body when the catheter is correctly positioned; a catheter segment extending from the head portion to a distal tip, the catheter segment having an exterior and a wall defining a passageway through the interior, the passageway defining at least three lumens, each lumen having a proximal end and a distal end and each extending from the head of the catheter at least a portion of the length from the head of the catheter to the distal end of the catheter; and an elongate sleeve attached to the catheter segment about the passageway so as to form an inflatable balloon which covers a portion of the exterior of the catheter segment, the elongate sleeve being in communication with one of the at least three lumens, the sleeve being in a collapsed state when not inflated wherein the sleeve closely surrounds the catheter segment, and wherein the sleeve extends radially outwardly from the catheter segment when inflated. Preferably, each of the at three lumens has one or more opening near the distal end of the lumen which provides for communication exterior of the catheter. This communication may, for example, be the excretion of fluids being passed through the head and lumens of the catheter which are intended to provide nutrition or medicine to a patient, the removal of fluids or gases from the patient through the catheter or the inflation and deflation of the retention mechanism which is secured to the catheter segment. Again, it is preferable that the attachment of the elongate sleeve to the exterior of the catheter segment occurs so as to form a proximal cuff and a distal cuff of the balloon. Further still, the catheter segment preferably further includes a stiff tip attached to the distal end of the catheter segment (opposite the head) and/or the catheter segment may also include an annular recess, wherein the sleeve is disposed in the annular recess when it is in a deflated state.

Another embodiment of the present invention defines a catheter including a head, the head comprising at least three openings through which fluid can be introduced, the head having a low profile; a catheter segment extending from the head, the catheter segment having a proximal end and a distal end, the catheter segment further including at least a first lumen, a second lumen, and a third lumen, each of the lumens having a proximal end and a distal end, and each lumen being in communication with at least one of the openings of the head; and a retention mechanism attached to the catheter segment at a position between the proximal end and the distal end of the segment. Preferably, the head of the catheter is designed such that the first one of the three openings in the head is adapted for communication with a first one of the three lumens in the catheter segment such that fluid communication may be achieved between the head and the jejunum of a patient; that a second one of the three openings in the head is adapted for communication with a second of the three lumens in the catheter segment such that fluid communication may be achieved between the head and a visceral organ of the patient; and a third one of the three openings in the head is adapted for communication with a third of the three lumens in the catheter segment such that communication may be achieved between the head and the retention mechanism. It is contemplated that the communication between the third opening in the head of the catheter and the retention mechanism may include, without limitation, fluid communication or triggering communication, depending on the type of retention mechanism used. That is, for example, where a balloon or elongate sleeve is retention mechanism the third opening in the head of catheter will preferably be adapted for fluid communication with the third lumen of the catheter. By way of further example, where the retention mechanism is triggerable (as discussed above) rather than inflatable, the third opening in the head need only be in or allow communication with the third lumen of the catheter (and ultimately the retention mechanism) which is sufficient to allow the retention mechanism to be triggered, released, activated or the like as well as to allow for retraction, deactivation or the like thereof. As way of an example of a suitable communication where a non-balloon or elongate sleeve retention mechanism is used, the third opening in the head of the catheter may be capable of accommodating a stylus which may pass through the opening and through or into the third lumen of the catheter so as to enable an operator to trigger or otherwise activate to enable the retention mechanism and/or to release or deactivate the retention mechanism to enable withdrawal thereof. As noted above the retention mechanism preferably may be a balloon and the balloon may be formed by a sleeve with a first end attached to the catheter segment so as to form a first cuff and a second end attached to the catheter segment so as to form a second cuff, the balloon collapsing on the catheter segment when not inflated to thereby facilitate insertion or removal.

The catheter segment of the present invention may also include a catheter shaft having a distal end and a stiff tip attached to the distal end of the catheter shaft. Preferably one or more of the openings in the head has a valve configured for selectively controlling fluid flow through one or more of the lumens. To reduce contamination of the catheter or of the patient the catheter may also include a protective covering for at least one of the openings. Any suitable protective covering may be used. Suitable protective coverings may include, for example, a cap, plug, or the like (as discussed in more detail above). Preferably the protective covering will also include a strap with at least one plug or the like for insertion into one or more of the openings. In a preferred embodiment, the proximal end of the catheter segment may include at least one external retention member or securing mechanism and a protective covering formed with the external retention member or securing mechanism, and wherein the external retention member includes a means for securing the protective covering to the catheter.

Further, as noted above with other embodiments, at least one of the openings of the head of the catheter may be capable of receiving an adapter. The adapter is preferably adapted for communication with one or more of the lumens of the catheter, wherein the adapter has a proximal end and a distal end and one or more lumens which allow fluid communication between the proximal end and the distal end of the adapter. In at least one embodiment of the present invention, at least one of the openings of the head of the catheter has a positioning mechanism capable of enabling proper alignment of the lumens in the catheter with one or more lumens in the adapter.

Additionally, in those embodiments having adapters or capable of receiving adapters, it is preferable for the catheter to have a means for releasably latching the adapter to the head of the catheter. Any suitable means for releasably latching or otherwise attaching an adapter to the head of the catheter is contemplated by the means for releasably latching. An exemplary means for releasably latching an adapter to the head of the catheter may include a leg extending from the distal end of the adapter, wherein the means further includes a finger formed at a free end of the leg and extending at a generally perpendicular angle relative to the leg.

Lastly, one or more embodiments of the catheter of the present invention may include a catheter segment having a radiopaque material (as discussed in more detail above), the material being positioned at one or more points along the catheter segment and/or at least one opening which corresponds to one or more of the openings at or near the distal end of the lumens, wherein fluid may be allowed to pass therethrough.

Whereas the invention has been shown and described in connection with a preferred embodiment thereof, it is understood that many modifications, additions, and substitutions may be made which are within the intended broad scope of the appended claims.

We claim:

1. A catheter comprising:
a head, the head comprising at least two openings through which fluid can be injected into or removed from a patient, the head having a low profile relative to a patient when the catheter is properly positioned in the patient;
a catheter segment extending from the head, the catheter segment having a proximal end and a distal end, the catheter segment further having at least a first lumen and a second lumen, each of the lumens having a proximal end and a distal end, and each lumen being in communication with at least one of the openings of the head; and
a retention mechanism attached to the catheter segment at a position between the proximal end and the distal end of the segment.

2. The catheter of claim 1, wherein the low profile head has three openings and wherein the catheter segment has three lumens.

3. The catheter of claim 2, wherein the low profile head has:
a first one of the three openings adapted for communication with a first one of the three lumens adapted for fluid communication between the low profile head and the jejunum of the patient;
a second one of the three openings adapted for communication with a second one of the three lumens adapted for fluid communication between the low profile head and a visceral organ of the patient; and
a third one of the three openings adapted for communication with a third one of the three lumens adapted for communication between the low profile head and the retention mechanism.

4. The catheter of claim 1, wherein the distal end of the catheter segment is adapted to terminate within a jejunum of a patient.

5. The catheter of claim 1, wherein the retention mechanism is a balloon.

6. The catheter of claim 5, wherein the balloon is formed by a sleeve with a first end attached to the catheter segment so as to form a first cuff and a second end attached to the catheter segment so as to form a second cuff, the balloon collapsing on the catheter segment when not inflated to thereby facilitate insertion or removal through a stoma.

7. The catheter according to claim 1, wherein the catheter segment comprises a catheter shaft having a distal end and a stiff tip attached to the distal end of the catheter shaft.

8. The catheter according to claim 1, wherein one or more of the openings in the low profile head has a valve configured for selectively controlling fluid flow through one or more of the lumens.

9. The catheter of claim 1 further comprising a protective covering for at least one of the openings.

10. The catheter of claim 9, wherein the protective covering comprises a cap.

11. The catheter of claim 9, wherein the protective covering comprises a strap with at least one plug for insertion into one or more of the openings.

12. The catheter of claim 11, wherein the proximal end of the catheter segment comprises at least one external retention member and a protective covering formed with the external retention member, and wherein the external retention member includes a means for securing the protective covering to the catheter.

13. The catheter of claim 1, wherein at least one of the openings of the low profile head is capable of receiving an adapter.

14. The catheter of claim 1 further comprising an adapter for communication with one or more of the lumens of the catheter, wherein the adapter has a proximal end and a distal end and one or more lumens which allow fluid communication between the proximal end and the distal end of the adapter.

15. The catheter of claim 13, wherein at least one of the openings of the low profile head has a positioning mechanism capable of enabling proper alignment of the lumens in the catheter with one or more lumens in the adapter.

16. The catheter of claim 13, wherein the catheter further includes a means for releasably latching the adapter to the low profile head of the catheter.

17. The catheter of claim 16, wherein the means for releasably latching comprises a leg extending from the distal end of the adapter, the means further comprising a finger formed at a free end of the leg and extending at a generally perpendicular angle relative to the leg.

18. The catheter of claim 1, wherein the catheter segment has at least one opening therein corresponding to one or more of the openings proximate the distal end of the lumens, wherein fluid may be allowed to pass therethrough.

19. The catheter of claim 1 further comprising a guide wire.

20. The catheter of claim 1 further comprising a radiopaque material, the material being positioned at one or more points along the catheter segment.

21. The catheter of claim 20, wherein the radiopaque material is tungsten or a tungsten containing material.

22. A balloon catheter adapted for placement through a stoma into a body cavity so that the balloon catheter is maintained in the stoma, the balloon catheter comprising:
a head having at least two openings through which a fluid can be passed, the head having a low profile relative to a patient when the catheter is correctly positioned within the patient;
a catheter segment extending from the head to a distal tip, the catheter segment having an exterior and a wall defining at least one passageway through an interior; and
an elongate sleeve attached to the exterior of the catheter segment about the passageway so as to form an inflatable balloon which covers a portion of the exterior of the catheter segment, the sleeve being in collapsed state when not inflated wherein the sleeve closely surrounds the catheter segment, and wherein the sleeve extends radially outwardly from the catheter segment when inflated, the sleeve being in communication with one of the openings in the head of the balloon catheter.

23. The balloon catheter of claim 22, wherein the passageway through the interior of the catheter segment includes a plurality of lumens, at least one of the lumens extending from the head of the catheter to a point at or near the distal end of the catheter segment, and at least another one of the lumens extending from the head of the catheter to the elongate sleeve.

24. The balloon catheter of claim 22 wherein the attachment of the elongate sleeve to the exterior of the catheter segment forms a proximal cuff and a distal cuff of the balloon.

25. The balloon catheter of claim 22, wherein the catheter segment comprises a catheter shaft and a stiff tip attached to a distal end of the catheter shaft, opposite the head.

26. The balloon catheter of claim 23, wherein one of the lumens therein is a feeding tube, the feeding tube having a proximal end and a distal end.

27. The balloon catheter of claim 26, wherein the distal end of the feeding tube is adapted to terminate within a jejunum of a patient.

28. The balloon catheter of claim 26, wherein one of the lumens is a venting lumen, wherein fluid contained within a visceral organ of a patient may be evacuated through the venting lumen.

29. The balloon catheter of claim 22, wherein one or more of the openings of the head of the catheter is capable of receiving an adapter.

30. A balloon catheter adapted for placement through a stoma into a body cavity so that the balloon catheter is maintained in the stoma, the balloon catheter comprising:
a low profile head having at least three openings through which a fluid may be injected, the head being low profile relative to a patient's body when the catheter is correctly positioned;
a catheter segment extending from the head to a distal end of the catheter, the catheter segment having an exterior and a wall defining a passageway through an interior, the passageway defining at least three lumens, each lumen having a proximal end and a distal end; and
an elongate sleeve attached to the catheter segment about the passageway so as to form an inflatable balloon which covers a portion of the exterior of the catheter segment, the elongate sleeve being in communication with one of the at least three lumens, the sleeve being in a collapsed state when not inflated wherein the sleeve closely surrounds the catheter segment, and wherein the sleeve extends radially outwardly from the catheter segment when inflated.

31. The balloon catheter of claim 30, wherein each of the at least three lumens extend from the head of the catheter at least a portion of the length to the distal end of the catheter.

32. The balloon catheter of claim 30, wherein the attachment of the elongate sleeve to the exterior of the catheter segment forms a proximal cuff of the balloon and a distal cuff of the balloon.

33. The balloon catheter of claim 30, wherein the catheter segment comprises a catheter shaft and a stiff tip attached to a distal end of the catheter shaft, opposite the head.

34. The catheter of claim 30, wherein the catheter segment comprise an annular recess, and wherein the sleeve is disposed in the annular recess.

35. A low profile transjejunal feeding system comprising:
a balloon catheter, the catheter having multiple lumens, a low profile head having at least two ports therein, and a balloon retention mechanism; wherein each of the lumens is in communication with at least one of the ports; and
a first adapter capable of attachment to one of the ports of the balloon catheter.

36. The system of claim 35 further comprising a second adapter capable of attachment to a second port of the balloon catheter.

37. The system of claim 35 further comprises a means for releasably latching the adapter to the head of the catheter.

38. The system of claim 37, wherein the means for releasably latching comprises a leg extending from the adapter, the means further comprising a finger formed at a free end of the leg and extending at a generally perpendicular angle relative to the leg.

39. The system of claim 35, further comprising an external retention member and a protective covering formed with the external retention member, and wherein the head further includes a means for securing the protective covering to the system.

40. The system of claim 39, wherein the external retention member and protective covering are integrally formed with the head of the system.

41. A catheter comprising:
a head, the head comprising at least two openings through which fluid can be introduced, the head having a low profile;
a catheter segment extending from the head, the catheter segment having a proximal end and a distal end, the catheter segment further having at least a first lumen and a second lumen, each of the lumens having a proximal end and a distal end, and each lumen being in communication with at least one of the openings of the head; and
a retention mechanism attached to the catheter segment at a position between the proximal end and the distal end of the segment.

42. The catheter of claim 41, wherein the head has three openings and wherein the catheter segment has three lumens.

43. The catheter of claim 42, wherein the head has:
a first one of the three openings adapted for communication with a first one of the three lumens adapted for fluid communication between the head and the jejunum of a patient;
a second one of the three openings adapted for communication with a second one of the three lumens adapted for fluid communication between the head and a visceral organ of the patient; and
a third one of the three openings adapted for communication with a third one of the three lumens adapted for communication between the head and the retention mechanism.

44. The catheter of claim 41, wherein the distal end of the catheter segment is adapted to terminate within a jejunum of a patient.

45. The catheter of claim 41, wherein the retention mechanism is a balloon.

46. The catheter of claim 45, wherein the balloon is formed by a sleeve with a first end attached to the catheter segment so as to form a first cuff and a second end attached to the catheter segment so as to form a second cuff, the balloon collapsing on the catheter segment when not inflated to thereby facilitate insertion or removal.

47. The catheter of claim 41, wherein the catheter segment comprises a catheter shaft having a distal end and a stiff tip attached to the distal end of the catheter shaft.

48. The catheter of claim 41, wherein one or more of the openings in the head has a valve configured for selectively controlling fluid flow through one or more of the lumens.

49. The catheter of claim 41 further comprising a protective covering for at least one of the openings.

50. The catheter of claim 49, wherein the protective covering comprises a cap.

51. The catheter of claim 49, wherein the protective covering comprises a strap with at least one plug for insertion into one or more of the openings.

52. The catheter of claim 51, wherein the proximal end of the catheter segment comprises at least one external retention member and a protective covering formed with the external retention member, and wherein the external retention member includes a means for securing the protective covering to the catheter.

53. The catheter of claim 41, wherein at least one of the openings of the head is capable of receiving an adapter.

54. The catheter of claim 41 further comprising an adapter for communication with one or more of the lumens of the catheter, wherein the adapter has a proximal end and a distal end and one or more lumens which allow fluid communication between the proximal end and the distal end of the adapter.

55. The catheter of claim 53, wherein at least one of the openings of the head has a positioning mechanism capable of enabling proper alignment of the lumens in the catheter with one or more lumens in the adapter.

56. The catheter of claim 53, wherein the catheter further includes a means for releasably latching the adapter to the head of the catheter.

57. The catheter of claim 56, wherein the means for releasably latching comprises a leg extending from the distal end of the adapter, the means further comprising a finger formed at a free end of the leg and extending at a generally perpendicular angle relative to the leg.

58. The catheter of claim 41, wherein the catheter segment has at least one opening therein corresponding to one or more of the openings proximate the distal end of the lumens, wherein fluid may be allowed to pass therethrough.

59. The catheter of claim 41 further comprising a guide wire.

60. The catheter of claim 41 further comprising a radiopaque material, the material being positioned at one or more points along the catheter segment.
